(19) United States
(12) Patent Application Publication
(10) Pub. No.: US 2025/0118449 A1
(43) Pub. Date: Apr. 10, 2025

(54) SYSTEMS AND METHODS FOR PATIENT CLASSIFICATION

(71) Applicant: GENFIT, LOOS (FR)

(72) Inventors: Yacine Hajji, Lille (FR); JÉRÉMY MAGNANENSI, LILLE (FR)

(21) Appl. No.: 18/378,471

(22) Filed: Oct. 10, 2023

(51) Int. Cl. G16H 50/70

(52) U.S. Cl. CPC G16H 50/70

(57) ABSTRACT

The present invention relates to a systems and methods to classify patients as eligible or not eligible to liver biopsy.

The present invention relates to a systems and methods to classify patients as eligible or not eligible to liver biopsy.

### BACKGROUND OF THE INVENTION

Metabolic dysfunction-associated steatohepatitis (MASH), formerly named nonalcoholic steatohepatitis or NASH, is a progressive condition that may lead to cirrhosis, hepatocellular carcinoma, and end-stage liver disease. It has now become a major indication for liver transplantation. Active steatohepatitis, defined histologically by a metabolic dysfunction-associated steatotic liver disease (MASLD) activity score (MAS)≥4 and significant fibrosis (fibrosis stage≥2), is commonly referred to as at-risk MASH as it is associated with a higher risk of liver-related mortality, liver cancer, and progression to cirrhosis (Harrison S A, et al., Lancet Gastroenterol Hepatol., 2020; 5 (11): 970-985). The risk of liver-related mortality increases exponentially with increase in fibrosis stage and mortality and morbidity rates increase exponentially once cirrhosis develops. Cirrhosis is the point where the liver is completely scarred and is beyond the self-healing ability.

In the absence of approved therapies, drug development for the treatment of at-risk MASH is an active area of research. Therefore, patients with at-risk MASH have to be acutely selected for clinical trials with histological endpoints, however, those with cirrhosis (F4) are excluded, as cirrhosis trials have specific designs and endpoints (Noureddin M, et al., Gastroenterology, 2020; 159:422-427).

Currently, inclusion of patients with at-risk MASH can only be performed through histopathological examination of a liver biopsy specimen. Nevertheless, liver biopsy has several recognized limitations including sampling errors, inter-observer variability, and hospitalization. The main disadvantage is the significant risk of complications including bleeding, pain and even death. Furthermore, a biopsy does not reflect the changes in the whole liver since it is a needle biopsy and the liver tissue obtained from the subcapsular region is not representative of the whole live. A biopsy does not differentiate early cirrhosis from progressed cirrhosis and therefore does not constitute a reliable prognostic predictor. In addition, the use of liver biopsy in clinical trials poses significant logistical challenges such as the cost of the procedure, and the screening failures rate (Sumida, Y et al., World J Gastroenterol, 2014; 20:475-485).

Therefore, there is a need to provide a screening method which could be carried out to select patients to be elected to liver biopsy. Indeed, quite often liver biopsy does not confirm at-risk MASH histologically, with a consequent increase in the screen failure rate.

The high screen failure rate observed with liver biopsy screening does not only mean unnecessarily exposing many patients to the risks associated with liver biopsy, but also increases the number of patients to be screened and the overall cost of the trial. Furthermore, if a screen failure rate is consistently high, it also carries the risk of demobilizing the investigative screening sites for a particular trial. Consequently, when conducting MASH clinical trials, it is essential to identify accurately patients with non-cirrhotic at-risk MASH, who will then undergo a liver biopsy for histological confirmation. So far, MASH clinical trials do not have non-invasive strategy for selecting patients to refer for liver biopsy other than relying on clinical and biological features that are neither sensitive nor specific.

In addition, since cirrhotic MASH patients are not the patients targeted by at-risk MASH treatments in development, it is also important and necessary to accurately diagnose non-cirrhotic at-risk MASH patients who will be receivers and responders of specific treatments.

Therefore, there is still an unmet medical need to develop a method to identify non cirrhotic at-risk MASH patients, for referral to liver biopsy in MASH clinical trials aiming to reduce the number of unnecessary procedures, to reduce the liver biopsy failure rate, to reduce the cost and duration of the screening process without inflating the Number Needed to Screen (NNS), and more generally to provide a method to diagnose more accurately MASH patients with fibrosis stage F2 or F3, targets of clinical trials and of therapies.

### DESCRIPTION OF THE INVENTION

The present invention is as represented in FIG. 1.

In one aspect, the invention relates to the provision of circulating levels of alanine aminotransferase (ALAT), aspartate aminotransferase (ASAT), platelets, hsa-miR-34a-5p and YKL-40, and the provision of the age and gender of a patient, analyzing the levels, age and gender to reference levels, age and gender, and based on said analysis, classifying said patient as a patient with MAS≥4, and F=2 or 3. This information can further be useful to assign the patient as eligible or not eligible to liver biopsy. Then, on the basis of the outcome of the biopsy analysis, the patient may be prescribed with a treatment of a liver condition, such as a treatment of MASH, or may be given lifestyle recommendations to treat his/her condition.

Circulating levels are determined from a sample of biological fluid of the patient. Such biological fluids include blood or a blood-derived sample, e.g. serum or plasma, or a sample of saliva, interstitial fluid or urine. In a particular embodiment, the sample is a sample of blood or a blood-derived sample, such as serum or plasma.

Illustrative methods for measuring circulating ALAT and ASAT include, without limitation, clinical measurement, cell culture methods and enzymatic assays. For example, the circulating levels of ALAT and ASAT may be measured by a blood test, such as a liver function test or liver enzyme test.

Circulating levels of platelets may be measured by medical imaging or a blood test, such as a complete blood count.

Circulating levels of YKL-40 may be measured by techniques such as immunoassays (e.g. ELISA (enzyme-linked immunosorbent assay), immunoturbidimetry, immuno-nephelometry, immune cytometry, protein array). Agents binding agents to YKL-40 useful in the practice of assays to determine the level of YKL-40 include, without limitation, antibodies, aptamers and peptides. In a particular embodiment, the binding agent is an anti-YKL antibody.

Circulating hsa-miR-34a-5p may be measured by quantitative PCR with specific primers and/or probes.

In one other aspect, the invention relates to a method for determining whether a patient is eligible or not eligible to liver biopsy. In a particular embodiment, the patient eligible to liver biopsy is defined as having a MASLD-Activity Score (MAS) greater or equal to 4, and fibrosis score of 2 or 3.

In a particular embodiment, the patient has MASH or is suspected of having MASH, as determined before implementation of the present invention.

In another aspect, the invention relates to a computer program comprising instructions that, when executed by a central processing unit, cause the central processing unit to assign a patient to a group of patients eligible or not eligible to liver biopsy. More specifically, the computer program may comprise instructions that, when executed by a central processing unit, cause the central processing unit to:
obtain the levels of ALAT, ASAT, platelets, hsa-miR-34a-5p and YKL-40, as determined from a sample of a patient;
obtain the age and the gender of the patient;
analyze the levels of the markers, the age and the gender of the patient; and
based on said analysis, assign the patient to a group of patients eligible to liver biopsy or to a group of patients not eligible to liver biopsy.

In a particular embodiment, the analysis made by the central processing unit comprises:
calculating a first score from the levels of ALAT, ASAT, Platelets and the age;
calculating a second score from the levels of hsa-miR-34a-5p and YKL-40 and the gender;
comparing the calculated first score to a first cutoff value; and
comparing the calculated second score to a second cutoff value.

In a particular embodiment, the first score (S1) is calculated with the following mathematical function: S1=age (years)×ASAT (U/L)/platelet (×10⁹/L)/√ALAT (U/L).

In a further particular embodiment, the second score (S2) is the following logistic function:

$$S 2 = \frac{1}{1 + exp ( - y )}$$

$$wherein$$

$$y = β0 + β 1 * log 10 ( miR - 34 a - 5 p ( Fold ) ) + β2 * log 10 ( Y K L - 40 ( ng / ml ) ) + β3 * Gender + β4 * log 10 ( m i R - 34 a - 5 p ( Fold ) ) * Gender$$

wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

In a further particular embodiment, the computer program causes the central processing unit to:
assign the patient to a group of patients eligible to liver biopsy if the first score is lower than the first cutoff value and the second score is higher than the second cutoff value; or
assign the patient to a group of patients not eligible to liver biopsy if the first score is higher than the first cutoff value or the second score is lower than the second cutoff value.

In a particular embodiment, the first cutoff is comprised between 2 and 3. In a more particular embodiment, the first cutoff is comprised between 2.4 and 2.8. In yet another embodiment, the first cutoff if equal to 2.48 or 2.67. In a particular embodiment, the first cutoff is equal to 2.48.

In a further particular embodiment, the second cutoff is comprised between 0.4 and 0.8. In another particular embodiment, the second cutoff is comprised between 0.5 and 0.7. In yet another embodiment, the second cutoff is equal to 0.53 or 0.68. In a particular embodiment, co2 is equal to 0.53.

According to another aspect, the present invention further provides a computer readable medium comprising the computer program described therein. According to a particular embodiment, the computer readable medium is a non-transitory computer-readable medium or a storage medium. In a particular embodiment, the storage medium is a RAM, a CD-ROM or a disk drive, such as a hard drive or a solid state drive.

According to another aspect, the invention relates to a system for patient classification, for use in determining whether said patient is eligible to liver biopsy with the aid of a digital computer, said system comprising:
a database;
at least one central processing unit; and
a memory comprising the computer program described herein.

In a particular embodiment, the database stores patients' data such as the levels of the markers discussed above, the age and the gender of the patients.

The invention will now be further illustrated with reference to the following examples, which should not be interpreted as limiting the scope of the invention to the specific methods and features disclosed therein.

### BRIEF DESCRIPTION OF THE FIGURES

FIG. 1 is a schematic view of the invention.

FIG. 2 is a further schematic view of the invention.

This disclosure also provides the following non-limiting embodiments:

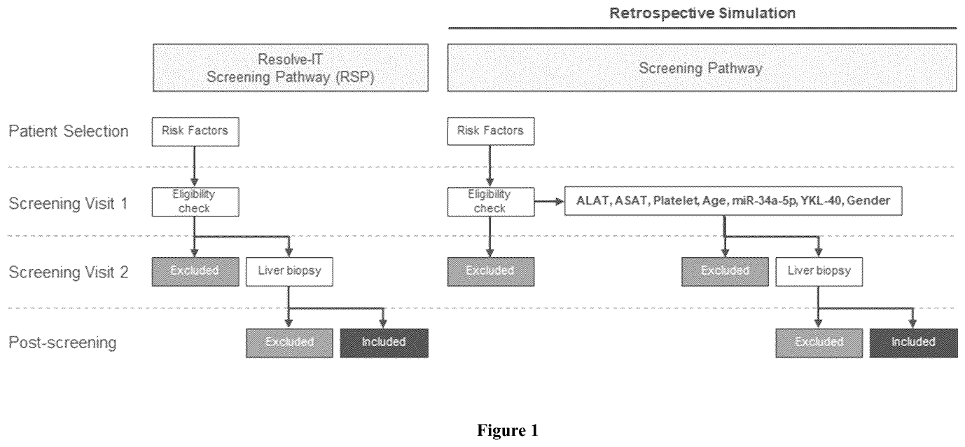

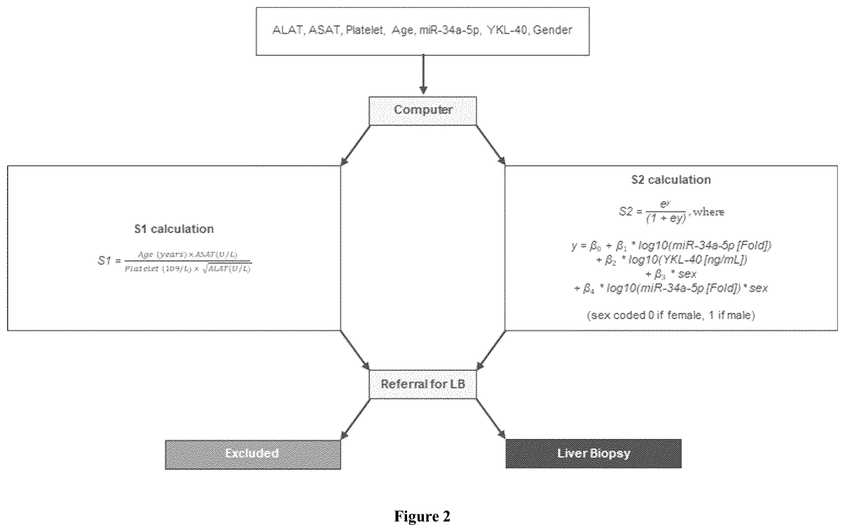

1. Invention as shown in FIG. 1 or FIG. 2, and described herein.

2. A computer program comprising instructions that, when executed by a central processing unit (CPU), cause the central processing unit to assign a patient to a group of MASH patients eligible or not eligible to liver biopsy, wherein the program cause the CPU to:
obtain the levels of ALAT, ASAT, platelets, hsa-miR-34a-5p and YKL-40, as determined from a sample of a patient;
obtain the age and the gender of the patient;
analyze the levels of the markers, the age and the gender of the patient; and
based on said analysis, assign the patient to a group of MASH patients eligible to liver biopsy or to a group of patients not eligible to liver biopsy.

3. The computer program according to embodiment 2, wherein the analysis made by the central processing unit comprises:
calculating a first score from the levels of ALAT, ASAT, Platelets and the age;
calculating a second score from the levels of hsa-miR-34a-5p and YKL-40 and the gender;
comparing the calculated first score to a first cutoff value; and
comparing the calculated second score to a second cutoff value.

4. The computer program according to embodiment 2 or 3, wherein the first score (S1) is calculated with the following mathematical function: S1=age (years)×ASAT (U/L)/platelet (×10⁹/L)/√ALAT (U/L).

5. The computer program according to embodiment 2 or 3 or 4, wherein the second score (S2) is calculated with the following logistic function:

$$S 2 = \frac{1}{1 + exp ( - y )}$$

$$wherein$$

$$y = β0 + β 1 * log 10 ( miR - 34 a - 5 p ( Fold ) ) + β2 * log 10 ( Y K L - 40 ( ng / ml ) ) + β3 * Gender + β4 * log 10 ( m i R - 34 a - 5 p ( Fold ) ) * Gender$$

wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

6. The computer program according to embodiment 3 or 4 or 5, wherein the first cutoff is comprised between 2 and 3.

7. The computer program according to embodiment 3 or 4 or 5 or 6, wherein the second cutoff is comprised between 0.4 and 0.8.

8. The computer program according to any one of embodiments 3 to 7, wherein the computer program causes the central processing unit to:
assign the patient to a group of patients eligible to liver biopsy if the first score is lower than the first cutoff value and the second score is higher than the second cutoff value; or
assign the patient to a group of patients not eligible to liver biopsy if the first score is higher than the first cutoff value or the second score is lower than the second cutoff value.

9. A computer readable medium comprising the computer program according to any one of embodiments 2 to 8.

10. A system for patient classification, for use in determining whether said patient is eligible to liver biopsy with the aid of a digital computer, said system comprising:
a database;
at least one central processing unit; and
a memory comprising the computer program according to any one of embodiments 2 to 8.

### EXAMPLES

### Example 1: Non-Invasive Tests as a Screening Method for Identification of Patients for Referral to Liver Biopsy in MASH Clinical Trials

Resolve-IT® is a randomized double-blind, placebo-controlled multicentre international phase 3 clinical trial (NCT02704403). Clinical data, blood samples and liver biopsy results were collected during the screening window. All liver biopsies were scored based on the MASH CRN classification scoring. Demographic and anthropometric characteristics (including bodyweight, age, sex and BMI), and medical history (Type-2 diabetes, dyslipidemia and hypertension) were collected at clinical sites from patients' medical records. Haematology and biochemical analyses were done by the central laboratory of the Resolve-IT® trial (BARC-Europe, Ghent, Belgium).

The levels of ALAT, ASAT, platelets, mir-34a-5p and YKL-40 were determined from serum samples of patients. The age and gender of the patients were also collected.

From these data, a first and second score were calculated.

The first score was calculated according to the following formula: S1=age (years)×ASAT (U/L)/platelet (×10⁹/L)/√ALAT (U/L).

The second score was calculated according to the following formula:

$$S 2 = \frac{1}{1 + exp ( - y )}$$

$$wherein$$

$$y = β0 + β 1 * log 10 ( miR - 34 a - 5 p ( Fold ) ) + β2 * log 10 ( Y K L - 40 ( ng / ml ) ) + β3 * Gender + β4 * log 10 ( m i R - 34 a - 5 p ( Fold ) ) * Gender$$

wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

Table 1 shows distribution of demography, comorbidity, blood dosage factors, histology and NITs values among the three groups of interest: not At-Risk MASH (patients who should not be included), At-Risk MASH (patients who should be included) and cirrhotic (patients who should not undergo a Liver Biopsy). The prevalence of at-risk MASH (without cirrhosis) was 40%. Descriptive results are shown through means±SD for numeric variables and proportion (sample size) for binary variables.

TABLE 1

| | Not-At-Risk | At-Risk | |
|---|---|---|---|
| | MASH | MASH^{a} | Cirrhotic |
| | (n = 1066) | (n = 765) | (n = 98) |
| Demographics | | | |
| Sex, male | 64 (682) | 56 (425) | 44 (43) |
| Age (years) | 53 ± 12 | 54 ± 12 | 58 ± 9 |
| Clinical | | | |
| characteristics | | | |
| T2DM | 34 (360) | 48 (365) | 64 (63) |
| Dyslipidemia | 46 (487) | 50 (385) | 42 (41) |
| Arterial | 52 (550) | 60 (458) | 69 (68) |
| hypertension | | | |
| BMI (kg/m²) | 33.39 ± 6.18 | 34.10 ± 6.09 | 33.61 ± 5.99 |
| Biochemistry | | | |
| ASAT (IU/L) | 31.06 ± 19.19 | 54.4 ± 36.22 | 56.41 ± 38.73 |
| ALAT (IU/L) | 44.72 ± 32.36 | 75.17 ± 53.89 | 61.20 ± 44.75 |
| GGT (IU/L) | 57.89 ± 71.21 | 85.11 ± 102.53 | 140.95 ± 144.82 |
| FPG (mmol/L) | 5.64 ± 1.50 | 6.27 ± 1.96 | 7.14 ± 2.78 |
| HbA1c (%) | 6.03 ± 0.94 | 6.39 ± 1.09 | 6.73 ± 1.21 |
| Triglycerides | 1.90 ± 1.07 | 2.04 ± 1.08 | 1.74 ± 0.79 |
| (mmol/L) | | | |
| Platelets | 249.04 ± 69.04 | 237.78 ± 65.53 | 201.66 ± 68.58 |
| (10e9/L) | | | |
| Histology | | | |
| F (0/1/2/3/4), | 28.0/54.0/6.6/ | 0/0/47.8/52.2/0 | 0/0/0/0/100 |
| % | 11.4/0 | | |
| MAS (0-1/2- | 25.2/36.3/ | 0/0/44.7/55.3 | 12.2/14.3/ |
| 3/4-5/6-8), % | 30.9/7.6 | | 37.8/35.7 |
| NITs | | | |
| S1 | 0.40 ± 0.24 | 0.70 ± 0.21 | 0.71 ± 0.21 |
| S2 | 1.11 ± 0.66 | 1.58 ± 0.95 | 2.29 ± 1.10 |

Values are expressed as mean ± SD or % (n), unless otherwise noted.
^{a}Without cirrhosis (F4).
BMI, body mass index;
F, fibrosis stage;
FPG, fasting plasma glucose;
GGT, gamma-glutamyl transferase;
HbA1c, hemoglobin A1c;
MAS, MASLD activity score;
MASH, metabolic-associated steatohepatitis;
NITs, non-invasive tests;
T2DM, type 2 diabetes mellitus

The comparison of not at-risk MASH group with at-risk MASH and cirrhotic patients in Table 1 demonstrates that both S1 and S2 values increase in the At-Risk MASH group: S1 increases from 0.4+/−0.24 in not at-risk MASH up to 0.7+/−0.21 in at-risk MASH and 0.71+/−0.21 in cirrhotic patients; S2 increases from 1.11+/−0.66 in not at-risk MASH up to 1.58+/−0.95 in at-risk MASH and 2.29+/−1.1 in cirrhotic patients.

These data show that, surprisingly, the first and second scores can advantageously be used in combination to classify patients as eligible to liver biopsy or not eligible to liver biopsy. Indeed, such patients are those with a MAS≥4, and F=2 or 3. It can be seen from the above results that while S2 is suitable to rule-in patients with MAS≥4, and F≥2, it cannot discriminate patients with F2 or F3 from F4. It can also be seen that the S1 score is suitable to rule-out patients with a fibrosis score equal to 4. Therefore, the combination of S1 and S2 is surprisingly suitable to achieve the identification of non-cirrhotic at risk MASH patients, and thus suitable to assign a patient as eligible to liver biopsy or not eligible to liver biopsy.

### Example 2: Screening of Patients to Liver Biopsy in MASH Clinical Trials

The selection of patients to be included in MASH clinical trial requires the accurate identification of patients to be enrolled. The aim is to reduce liver biopsy failure rate (LBFR), the number of cirrhotic patients to avoid associated risk with these specific patients and the associated cost.

In order to compare the Resolve-IT Screening Pathway (RSP) with the screening method of the invention, estimations were performed with respect to proportions of patients who would have ended up in each exclusion/inclusion group at the end of the screening. 1929 patients with available S1 and S2 scores, demographic and anthropometric data, and liver biopsies were analyzed.

The potential of the screening method of the invention before sending patients to liver biopsy was analyzed and compared to the RSP.

For cost estimate, in Resolve-IT®, 4 groups of costs generated invoices: Screening Visit procedures, S1 measurement, liver biopsy and liver biopsy preparation.

The results are shown in Table 2.

TABLE 2

| | Resolve-IT | | | | |
|---|---|---|---|---|---|
| | Screening | | | | |
| | Pathway | | | | |
| | (RSP) | S1 and S2 screening method | | | |
| S1 cutoff | — | <2.67 | <2.48 | <2.67 | <2.48 |
| S2 cutoff | — | ≥0.53 | ≥0.53 | ≥0.68 | ≥0.68 |

Performances

| | | | | | |
|---|---|---|---|---|---|
| Sensitivity | — | 0.78 | 0.77 | 0.60 | 0.59 |
| Specificity | — | 0.69 | 0.69 | 0.83 | 0.84 |
| LR+ | — | 2.5 | 2.5 | 3.6 | 3.7 |
| LR− | — | 0.3 | 0.3 | 0.5 | 0.5 |
| NNS | 3220 | 4630 | 4792 | 6023 | 6252 |
| LBs performed, n | 2522 | 1648 | 1646 | 1450 | 1442 |
| LBFR (n) | 60% | 39% | 39% | 31% | 31% |
| | (1522) | (648) | (646) | (450) | (442) |
| Failures avoided vs | | 874 | 876 | 1072 | 1080 |
| RSP, n (% reduction) | — | (−57%) | (−58%) | (−70%) | (−71%) |
| Number of cirrhotic | 128 | 98 | 89 | 93 | 81 |
| patients referred | | (−23%) | (−30%) | (−27%) | (−37%) |
| for LB | | | | | |
| Total cost of | 15.0 | 13.6 | 13.9 | 14.9 | 15.2 |
| screening ($US M) | | (−9%) | (−7%) | (−1%) | (+1%) |

Sensitivity: the proportion of positive patients (non-cirrhotic at-risk MASH) detected by the tested method during patients referral before Liver Biopsy; Specificity: the proportion of negative patients (either not-at-risk MASH or cirrhotic) detected by the tested method during patients referral before Liver Biopsy; LBFR: Liver Biopsy Failure Rate; NNS: Number of patients Needed to Screen; LB: Liver Biopsy; RSP, RESOLVE-IT screening pathway, LR+: positive likelihood ratio, which is defined as the probability of a person who has the disease testing positive (non-cirrhotic at-risk MASH) divided by the probability of a person who does not have the disease testing positive (either not-at-risk MASH or cirrhotic); LR−: negative likelihood ratio, which is defined as the probability of a person who has the disease testing negative (non-cirrhotic at-risk MASH) divided by the probability of a person who does not have the disease testing negative (either not-at-risk MASH or cirrhotic); Positive liver biopsies were set at 1000.

The LBFR decreases from 60% with the RSP pathway to 39% with the method of the invention with S2 cut-off set at 0.53 and S1 cut-off set at 2.48 or 2.67. The LBFR could be further reduced by using higher S2 cut-off value (0.68), but at the cost of an increased Number of patients Needed to Screen (NNS) from 4630 patients (cut-off S1<2.67, cut-off S2≥0.53) and 4792 patients (cut-off S1<2.48, cut-off S2≥0.53) up to 6023 patients (cut-off S1<2.67, cut-off S2≥0.68) and 6252 patients (cut-off S1<2.48, cut-off S2≥0.68).

The screening method with the combination of the S1 and S2 scores, with a S1 cut-off<2.48 and a S2 cut-off≥0.53 achieved a greater reduction in the number of cirrhotic patients referred for liver biopsy (−30%) and a 7% decreased cost compared to the usual screening pathway in Resolve-IT® study.

To demonstrate that this screening pathway method does not introduce bias in the selection of patients included in MASH clinical trial, patient characteristics were compared between the screening pathways: RSP and the screening methods with S1 and S2 scores (Table 3).

TABLE 3
Characteristics of patients included with NITs screening methods

| | | S1 and S2 |
|---|---|---|
| | | screening |
| | RSP | method |
| S1 cutoff | — | <2.48 |
| S2 cutoff | — | ≥0.53 |
| Age (years) | 54 ± 12 | 53 ± 12 |
| T2DM | 47.7 | 47.9 |
| Sex, male | 55.6 | 54.7 |
| F ≥ 3 | 52.2 | 54.1 |
| MAS ≥ 6 | 55.3 | 58.6 |

Values are expressed as mean ± SD, or %.
F, fibrosis stage;
MAS, metabolic dysfunction-associated steatotic liver disease (MASLD) activity score;
RSP, RESOLVE-IT screening pathway;
T2DM, type 2 diabetes mellitus

In conclusion, in clinical trials the method of the invention has the potential to significantly improve the recruitment process by reducing significantly the LBFR, increasing the proportion of positive liver biopsy, decreasing the number of F4 patients referred to liver biopsy and reducing the screening cost with a manageable Number of patients Needed to be Screened. Furthermore, no selection bias was observed in patients included with the sequential pathway vs the Resolve-IT® Standard Procedure regarding Type 2 Diabetes Mellitus 10 prevalence, percentage of males, or histology which represent randomization parameters at inclusion.

1. Invention as shown in FIG. 1 or FIG. 2, and described herein.

2. A computer program comprising instructions that, when executed by a central processing unit (CPU), cause the central processing unit to assign a patient to a group of MASH patients eligible or not eligible to liver biopsy, wherein the program cause the CPU to:
obtain the levels of ALAT, ASAT, platelets, hsa-miR-34a-5p and YKL-40, as determined from a sample of a patient;
obtain the age and the gender of the patient;
analyze the levels of the markers, the age and the gender of the patient; and
based on said analysis, assign the patient to a group of MASH patients eligible to liver biopsy or to a group of patients not eligible to liver biopsy.

3. The computer program according to claim 2, wherein the analysis made by the central processing unit comprises:
calculating a first score from the levels of ALAT, ASAT, Platelets and the age;
calculating a second score from the levels of hsa-miR-34a-5p and YKL-40 and the gender;
comparing the calculated first score to a first cutoff value; and
comparing the calculated second score to a second cutoff value.

4. The computer program according to claim 3, wherein the first score (S1) is calculated with the following mathematical function: S1=age (years)×ASAT (U/L)/platelet (×10⁹/L)/√ALAT (U/L).

5. The computer program according to claim 3, wherein the second score (S2) is calculated with the following logistic function:
$$S 2 = \frac{1}{1 + exp ( - y )}$$
$$wherein$$
$$y = β0 + β 1 * log 10 ( miR - 34 a - 5 p ( Fold ) ) + β2 * log 10 ( Y K L - 40 ( ng / ml ) ) + β3 * Gender + β4 * log 10 ( m i R - 34 a - 5 p ( Fold ) ) * Gender$$
wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

6. The computer program according to claim 4, wherein the first cutoff is comprised between 2 and 3.

7. The computer program according to claim 5, wherein the second cutoff is comprised between 0.4 and 0.8.

8. The computer program according to claim 3, wherein the computer program causes the central processing unit to:
assign the patient to a group of patients eligible to liver biopsy if the first score is lower than the first cutoff value and the second score is higher than the second cutoff value; or
assign the patient to a group of patients not eligible to liver biopsy if the first score is higher than the first cutoff value or the second score is lower than the second cutoff value.

9. A computer readable medium comprising the computer program according to claim 2.

10. A system for patient classification, for use in determining whether said patient is eligible to liver biopsy with the aid of a digital computer, said system comprising:
a database;
at least one central processing unit; and
a memory comprising the computer program according to claim 2.
